(19) United States
(12) Patent Application Publication
(10) Pub. No.: US 2026/0027299 A1
(43) Pub. Date: Jan. 29, 2026

(54) SYSTEM AND METHOD FOR MANAGING PATIENT HYDRATION

(71) Applicant: CareFusion 303, Inc., San Diego, CA (US)

(72) Inventors: Claire Ellen Knight, Arlington, TX (US); Michael K. Workman, Carlsbad, CA (US); Lisa Diggett, Overland Park, KS (US)

(21) Appl. No.: 18/993,768

(22) PCT Filed: Jul. 22, 2022
(86) PCT No.: PCT/US2022/038071

§ 371 Date: Jan. 13, 2025

(51) Int. Cl. A61M 5/172; G16H 20/17; G16H 40/63

(52) U.S. Cl. CPC A61M 5/1723; G16H 20/17; G16H 40/63; A61M 2205/3303

(57) ABSTRACT

A method for managing patient hydration is provided. The method includes receiving a fluid delivery request that includes a first programmed amount of a first fluid to be provided by a first medical device. Additionally, the method includes identifying a second programmed amount of a second fluid provided by or to be provided by a second medical device. Further, the method includes determining that a medical device operation should be adjusted based on the first and second programmed amounts. Finally, the method includes adjusting the medical device operation responsive to the determination and based on the fluid delivery request and the first and second programmed amounts.

### BACKGROUND

Fluid overload, also known as hypervolemia, occurs when someone has too much fluid in their body. It can cause cramping, headaches, bloating, high blood pressure, and even heart failure. It is a common concern in infusion therapy, especially when someone receives liquids from multiple sources (e.g., multiple infusion pumps). When someone receives liquids from multiple sources, and particularly when those sources provide liquids at different rates and over different time periods, it becomes increasingly difficult for a medical professional (e.g., a nurse, an operator, etc.) to keep track of everything. Thus, there is a significant risk of fluid overload in infusion therapy. On the other hand, too little fluid may lead to dehydration. Busy clinical environments, understaffing, and the like make it increasingly difficult to monitor when fluid sources are depleted, and thus appropriate fluid intake may be overlooked. Dehydration from lack of fluid intake can cause cramping, headaches, dizziness, and low blood pressure and, in critical settings, may destabilize the patient. Accordingly, fluid deprivation during an infusion therapy is also important to avoid.

Accordingly, there is a need for efficient monitoring and management of a patient's hydration levels, especially with regards to patients undergoing infusion therapy.

### SUMMARY

According to various aspects of the subject technology, a method for managing patient hydration includes receiving a fluid delivery request that includes a first programmed amount of a first fluid to be provided by a first medical device; identifying a second programmed amount of a second fluid provided by or to be provided by a second medical device; determining, based on the first and second programmed amounts, that a medical device operation should be adjusted; and adjusting, responsive to the determination, the medical device operation based on the fluid delivery request and the first and second programmed amounts.

According to various aspects of the subject technology, a system includes a first medical device, a second medical device, and an electronic device that includes one or more processors and memory storing one or more programs, the one or more programs including instructions, which, when executed by the one or more processors, cause the electronic device to perform the aforesaid method of this section.

According to various aspects of the subject technology, a non-transitory, computer-readable storage medium stores one or more programs that include instructions which, when executed by the one or more processors of an electronic device, cause the electronic device to perform the aforesaid method of this section.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the Figures. Like reference numerals refer to corresponding parts throughout the figures and description.

FIG. 1 depicts an example infusion device, according to various aspects of the subject technology.

FIG. 2 depicts an example user interface, according to various aspects of the subject technology.

FIG. 3 depicts a first example process for managing patient hydration, according to various aspects of the subject technology.

FIG. 4 depicts a second example process for managing patient hydration, according to various aspects of the subject technology.

FIG. 5 depicts a conceptual diagram illustrating an example electronic system for managing patient hydration, according to various aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

A hydration management system can source data from medical devices (e.g., an infusion pump, a smart drip, a dialysis machine, a catheter system, a fluid monitoring system, a specialized patient monitoring device, etc.) in order to determine the current and projected hydration levels of a patient. The system can monitor patient hydration in order to, for example, ensure patient hydration levels never fall below or rise above unsafe limits. The system can also receive fluid administration requests and respond to said requests based on the aforesaid hydration levels. For example, if the patient is at risk of fluid overload, the system may deny the request, modify the request, or adjust the operation of a medical device that is already administering fluid to the patient.

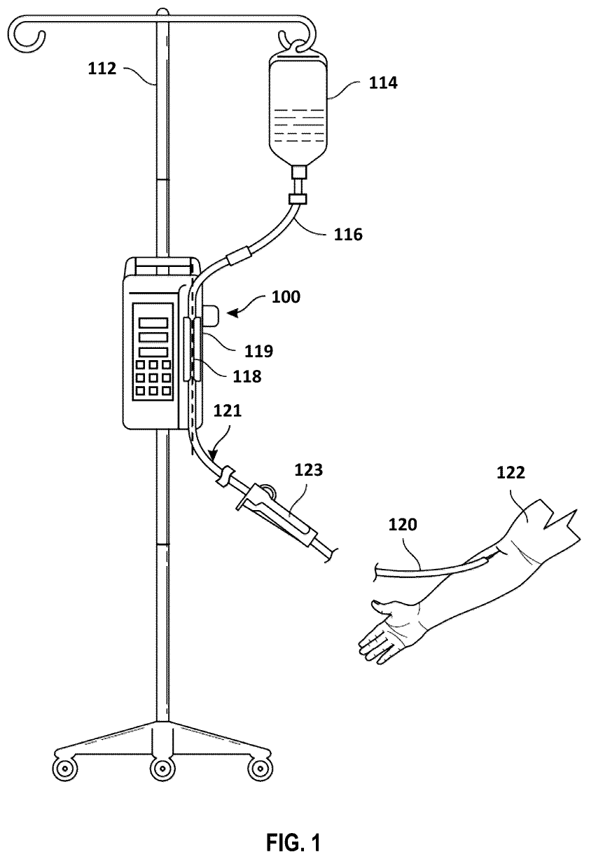

FIG. 1 depicts an example infusion device 100 (e.g., an infusion pump), shown in use in its intended environment, according to various aspects of the subject technology. In particular, the infusion device 100 is shown mounted to an intravenous (IV) pole 112 on which a fluid source 114 containing a fluid is held. The fluid source 114 is connected in fluid communication with an upstream portion 116 of a fluid infusion line 121. The fluid infusion line 121 is a conventional IV infusion type tube typically used in a hospital or medical environment and is made of any type of flexible tubing appropriate for use to infuse therapeutic fluids into a patient, such as silicone. A flexible portion 118 of the fluid infusion line 121 is mounted in operative engagement with a peristaltic pumping mechanism 119, for propelling fluid through a downstream portion 120 of the fluid infusion line 121, for example, to a patient's arm 122.

It should be understood that the upstream portion 116, the flexible portion 118, and the downstream portion 120 of the fluid infusion line 121 may be portions of the same continuous flexible tubing, with the portions defined by their position relative to the peristaltic pumping mechanism 119. For convenience, the continuous length of flexible tubing is indicated referred to herein as the fluid infusion line 121. A roller clamp 123 (e.g., configured to provide for mechanical compression of the line to block the flow) may be positioned on the downstream portion 120 of the fluid infusion line 121 between the infusion device 100 and the patient's arm 122. In this context, the term “upstream” refers to that portion of the flexible tubing that extends between the fluid source 114 and the peristaltic pumping mechanism 119, and the term “downstream” refers to that portion of the flexible tubing that extends from the peristaltic pumping mechanism 119 to the patient's arm 122.

It should also be understood that multiple medical devices (e.g., in addition to infusion device 100) can concurrently administer fluids to the same patient (e.g., to the patient's arm 122). In some implementations, multiple medical devices, such as infusion device 100, concurrently administer fluids to the patient. For example, in some implementations, the patient receives fluids from multiple different infusion devices, such as infusion device 100. As another example, in some implementations, the patient receives fluids from a smart drip, a dialysis machine, and/or a feeding tube.

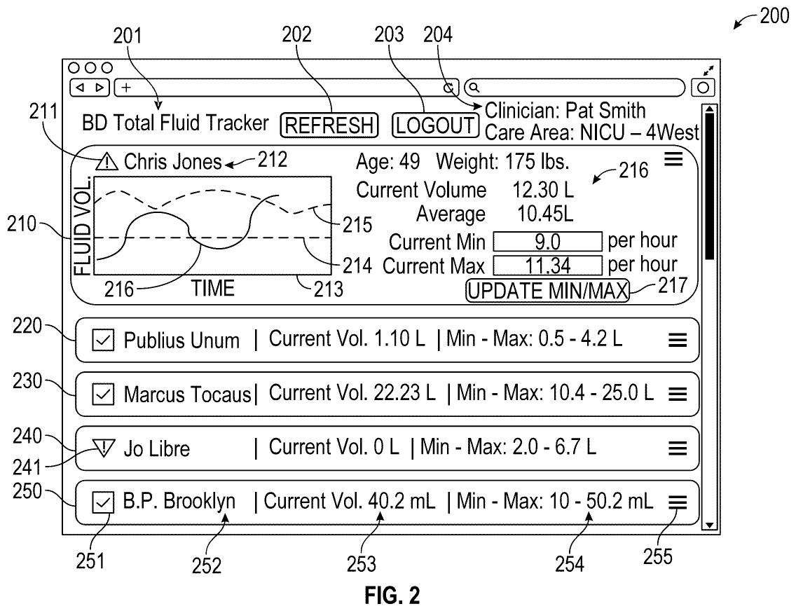

FIG. 2 depicts an example user interface 200, according to various aspects of the subject technology. According to various implementations, the user interface 200 is displayed by an infusion device, such as infusion device 100 of FIG. 1. In some implementations, the user interface 200 is displayed on a computing device (e.g., a computer, a smartphone, a tablet, etc.), or a device associated with a dispensing machine (e.g., a medication dispensing machine). In such implementations, the user interface may be operably connected to the infusion device 100 via the healthcare organization's electronic network.

As depicted in FIG. 2, in some implementations, the user interface 200 includes a title 201, a refresh button 202, a logout button 203, and device information 204. While not specifically illustrated, the title 201 may identify the device that displays the user interface 200, or the device or devices to which the user interface is operably connected (e.g., the infusion device 100). The refresh button 202 may allow a user (e.g., a medical professional, an operator, etc.) to manually refresh the user interface 200 and/or cause the system to obtain (and display) updated data from connected devices (e.g., infusion device(s) 100). The logout button 203 may allow a current user to log out of an account associated with the user interface 200 and/or the operably connected device(s). For example, in some implementations, the user interface 200 displays patient information (e.g., a hydration of a patient, etc.) that may be protected by requiring a user to log in in order to access the information. After using the device, the user can log out via the logout button 203 in order to protect the information. The device information 204 may further include information identifying the physician currently logged in to the device (e.g., “Pat Smith”). The device information 204 may also include information regarding the care area at which the device that displays the user interface 200 is being used (e.g., “NICU-4West”).

As in the depicted example, the user interface 200 includes patient information dashboards 210, 220, 230, 240, and 250 for respective patients. In some implementations, patient information dashboards may be displayed in an expanded view, such as with patient information dashboard 210. In some implementations, patient information dashboards may be displayed in a collapsed view, such as with patient information dashboards 220, 230, 240, and 250. In some implementations, an expand button allows a user to expand or collapse a patient information dashboard. For example, patient information dashboard 250 is depicted in FIG. 2 as displayed in a collapsed view. Patient information dashboard 250 includes an expand button 255 that, when selected, may cause the patient information dashboard 250 to display in an expanded view.

According to various implementations, the patient information dashboards 210, 220, 230, 240, and 250 are selectable for further expansion or reporting purposes. For example, dashboard 210 is shown in an expanded state. As will be further described, each dashboard (whether expanded or not) may include a hydration status indicator (e.g., hydration status indicators 211, 241, or 251). The hydration status indicator indicates whether the patient is at risk of fluid overload or deprivation. For example, hydration status indicator 211 of patient information dashboard 210 may indicate that the patient associated with patient information dashboard 210 is at risk of fluid overload. In some implementations, the hydration status indicator indicates that a patient is at risk of dehydration. For example, in the depicted example, hydration status indicator 241 of patient information dashboard 240 indicates that the patient associated with patient information dashboard 240 is at risk of dehydration. In some implementations, a hydration status indicator indicates that a patient is neither at risk of fluid overload nor at risk of dehydration (i.e., the patient is properly hydrated). For example, hydration status indicator 251 of patient information dashboard 250 indicates that the patient associated with patient information dashboard 250 is properly hydrated.

Each patient information dashboard 210, 220, 230, 240, and 250 includes a patient name 212 (e.g., “Chris Jones”) associated with the patient for which data is displayed by the dashboard. For example, patient information dashboard 250 includes patient name 252 (i.e., “B.P. Brooklyn”).

In some implementations, the patient information dashboard includes a patient hydration graphic (e.g., patient hydration graphic 213). In the depicted example, the patient hydration graphic is a graph with an x-axis and a y-axis. For example, patient hydration graphic 213 has an x-axis that corresponds to time (e.g., measured in minutes, hours, etc.), and a y-axis that corresponds to fluid volume (e.g., measured in liters, etc.). Other implementations may include different types of graphs such as bar charts, pie diagrams, and the like.

In some implementations, patient hydration graphic 213 includes one or more threshold indicators 214 and 215. In some implementations, a threshold indicator indicates a fluid delivery threshold. For example, the depicted threshold indicator 214 may indicate a minimum fluid delivery threshold that represents a risk of patient dehydration. For example, the threshold may indicate a level of hydration at which the patient is at risk of dehydration. The threshold indicator 214 may alternatively indicate a threshold representative of fluid overload, or excess hydration.

In some implementations, the patient hydration graphic 213 includes a variable threshold. For example, the depicted dotted-line threshold indicator 215 may represent a dynamically-estimated patient hydration threshold based on patient physiological parameters (e.g., weight, BMI, sex, age, hydration level, dehydration level, urine test result (e.g., volume, pH level, analyte content (such as salt), etc.), breathalyzer, or other measured value of the patient). The parameters may be received from a sensor, received from a patient medical record, or derived from information received from such sources. Using one or more of the parameters, a hydration level analysis process may be selected and, based on processing one of more of the parameters with the hydration level analysis process, generate a dynamic threshold value. While hydration threshold 215 is depicted as being dynamic, the threshold 215 may be static in some implementations.

Patient hydration graphic 213 includes a time-varying waveform 216 indicative of the patient's current hydration level (e.g., measured via urine sample, etc.). As data is collected by the system, this waveform 216 (and/or dynamic threshold 215) may update according to time. Where the threshold indicator 215 is a maximum fluid delivery threshold that represents a risk of fluid overload, the named patient (i.e., “Chris Jones”) may currently be at risk of fluid overload because his current hydration level is above the maximum fluid delivery threshold 215. Accordingly, the hydration status indicator 211 may indicate that the patient is currently at risk of fluid overload.

According to various implementations, patient information 216, including the age and/or weight of the patient, is also displayed. In the depicted example, the information includes a current volume associated with the patient (e.g., a current amount of fluid in the body of the patient, a current volume of fluid being administered to the patient over a certain period of time, etc.), an average volume associated with the patient (e.g., an average amount of fluid provided to the patient over a certain amount of time, etc.), a current minimum amount of fluid (e.g., a minimum fluid delivery threshold) associated with an amount of time (e.g., “per hour”), and a current maximum amount of fluid (e.g., a maximum fluid delivery threshold) associated with an amount of time (e.g., “per hour”). In the depicted example, the patient information dashboard 210 further includes an update button 217 that allows a user to update the minimum or maximum thresholds 214 and 216. The threshold input by the user may override the dynamic threshold. For example, if a medical condition of the patient (e.g., the patient is diabetic, the patient is undergoing chemotherapy, etc.) increases the importance of ensuring the patient is not dehydrated and/or fluid overloaded, the user may adjust the minimum or maximum amounts of fluid (e.g., increase the minimum fluid delivery threshold, decrease the maximum fluid delivery threshold, etc.) in order to account for the medical condition of the patient.

In some implementations, the patient hydration graphic 213, the patient information 216, and the update button are visible 217 while the patient information dashboard is displayed in an expanded view (e.g., they are not visible while the patient information dashboard is displayed in a collapsed view). For example, patient information dashboard 210 is displayed in an expanded view, and the patient hydration graphic 213, the patient information 216, and the update button 217 of the patient information dashboard 210 are visible. By contrast, patient information dashboard 250 is displayed in a collapsed view, and a respective patient hydration graphic, patient information, and update button associated with the patient information dashboard 250 are not visible.

In some implementations, the hydration status indicator, the patient's name, a portion of the patient information, and the expand button (e.g., expand button 255) are visible while the patient information dashboard is displayed in a collapsed view (e.g., they are visible regardless of whether the patient information dashboard is displayed in an expanded or a collapsed view). For example, patient information dashboard 250 is displayed in a collapsed view, and the hydration status indicator 251, the patient name 252, the current volume 253 (e.g., a current amount of fluid in the body of the patient, a current volume of fluid being administered to the patient over a certain period of time, etc.), maximum and minimum amounts of fluid 254 (e.g., a minimum fluid delivery threshold, a maximum fluid delivery threshold, etc.), and the expand button 255 of the patient information dashboard 250 are visible.

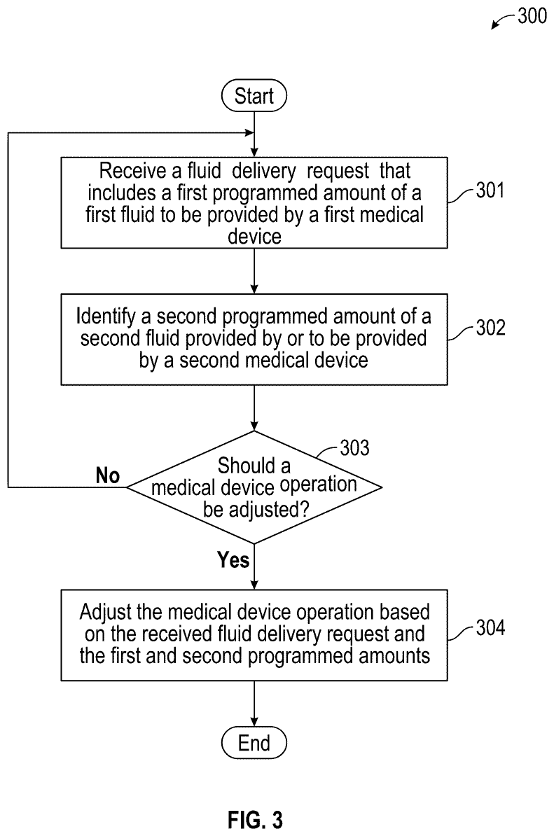

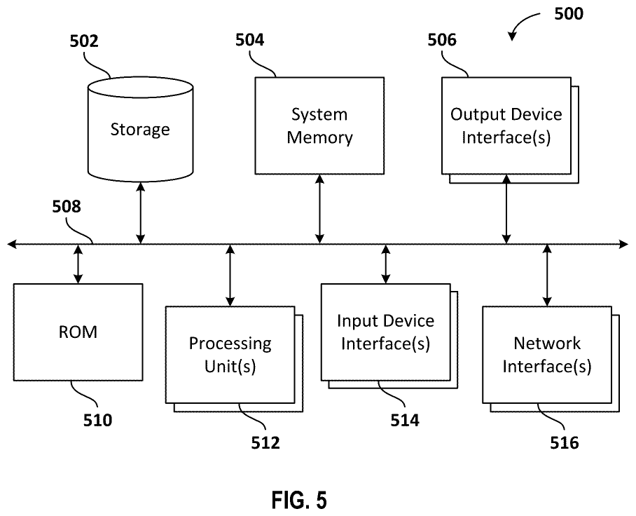

FIG. 3 depicts a first example process 300 for managing patient hydration, according to aspects of the subject technology. One or more blocks of process 300 may be implemented, for example, by one or more computing devices, such as the infusion device 100 of FIG. 1 or the processing unit(s) 512 of FIG. 5. In some implementations, one or more of the blocks may be implemented based on one or more machine learning algorithms. In some implementations, one more of the blocks may be implemented apart from other blocks, and by one or more different processors or devices. Further, for explanatory purposes, the blocks of example process 300 are described as occurring in serial, or linearly. However, multiple blocks of example process 300 may occur in parallel. Additionally, the blocks of example process 300 need not be performed in the order shown and one or more of the blocks of example process 300 need not be performed.

As depicted in FIG. 3 the process 300 may include receiving (301) a fluid delivery request that includes a first programmed amount of a first fluid to be provided by a first medical device. The amount of fluid may be representative of an amount of fluid to be administered per unit of time (e.g., 1051 ml per hour) or may represent an amount of fluid already administered or administered within a predetermined prior window of time (e.g., during the last 30 or 60 minutes). The request may originate from an infusion pump as the result of a clinician programming the pump to start an infusion of the fluid. In some implementations, the fist medical device may be an automated dispensing machine (ADM) and the request may be a request to dispense the first fluid (e.g., in the first programmed amount) from the ADM. According to the various examples herein, the request may be electronically received by the system responsible for implementation of the process 300. For example, a computing device (e.g., a server) may be operably connected via a network to the disclosed first and second medical devices, receive fluid delivery requests and other programmed amounts from those devices, and make determinations as to whether one or more of medical devices should be adjusted as described herein.

In some implementations, by the time the fluid delivery request is received (301), the first medical device is already providing, or has already provided (e.g., dispensed if by an ADM), the first fluid. In some implementations, the fluid delivery request further includes a medication order for delivery of a medication to a patient by the first medical device. This order may be received into the system, for example, by a pharmacist at a pharmacy system terminal of the healthcare organization. The first programmed amount may include, for example, a volume of the first fluid (e.g., measured in milliliters) and/or an amount of a medication (e.g., 400 milligrams dopamine). In some implementations, the first and/or second fluid includes an intravenous fluid (e.g., a saline solution, a Ringer's solution, etc.). In some implementations, the first and/or second fluid includes a medication (e.g., vancomycin, hydromorphone, etc.).

In some implementations, the first medical device includes a first infusion pump configured to provide the first programmed amount of the first fluid over a first programmed period of time. In some implementations, adjusting the medical device operation includes adjusting the first programmed period of time. For example, the first infusion pump may be configured to provide the first programmed amount of the first fluid over a period of sixty minutes. However, the first programmed period of time may be adjusted by being increased from sixty minutes to ninety minutes (e.g., by delaying further administration of the first fluid for another thirty minutes, or by decreasing the rate at which the first fluid is provided).

As depicted in FIG. 3, the process 300 may include identifying (302) a second programmed amount of a second fluid provided by or to be provided by a second medical device. For example, the system may communicate (e.g., via Wi-Fi, Bluetooth, etc.) with the second medical device via the healthcare organization's electronic network (e.g., via the network interface(s) 516 of FIG. 5). In some implementations, identifying (302) the second programmed amount includes receiving the second programmed amount from the second medical device. In some implementations, identifying (302) the second programmed amount includes looking up an amount of fluid infused or to be infused by a device in a database, such as the storage 502 of FIG. 5. For example, the second programmed amount may be received from an electronic medical records (EMR) system to which the amount was reported by the medical device or an associated clinician.

In some implementations, the second medical device has already provided the second programmed amount of the second fluid, or has provided some of the second programmed amount of the second fluid, and is programmed to continue to provide the second fluid until the second medical device has provided the entirety of the second fluid. In yet other implementations, the second medical device has not yet provided any of the second fluid, but the second medical device is designated or programmed to provide the second programmed amount of the second fluid at a future point in time.

In some implementations, the second programmed amount includes a volume of the second fluid (e.g., measured in milliliters). In some implementations, the second programmed amount includes an amount of a medication (e.g., 400 milligrams dopamine). In some implementations, the second fluid includes an intravenous fluid (e.g., a saline solution, a Ringer's solution, etc.). In some implementations, the second fluid includes a medication (e.g., vancomycin, hydromorphone, etc.).

In some implementations, the second medical device includes a second infusion pump configured to provide the second programmed amount of the second fluid over a second programmed period of time. In some implementations, adjusting the medical device operation includes adjusting the second programmed period of time. For example, the second infusion pump may be configured to provide the second programmed amount of the second fluid over a period of forty-five minutes. However, the second programmed period of time may be adjusted by being decreased from forty-five minutes to thirty minutes (e.g., by increasing the rate at which the second fluid is provided).

The example process 300 includes determining (303) whether a medical device operation should be adjusted. As will be described further, the medical operation to be adjusted may include, for example, adjusting the amount of fluid per unit of time that an infusion device 100 administers or is programmed to administer to a patient. Medical device operation may include an alert mechanism by which the adjustment includes displaying an alert. In the case of an ADM, the medical device operation may be a dispense of a medication or fluid, and the determining (303) may include determining whether the dispense should be allowed or prevented, or whether the amount dispensed should be increased or decreased.

According to various implementations, the determination (303) made by the system is based on the first and second programmed amounts. For example, the system may determine that the medical device operation should be adjusted based on determining that a sum of the first and second programmed amounts satisfies a fluid delivery threshold (see, e.g., threshold indicator 214 of FIG. 2). In some implementations, that the sum of the first and second programmed amounts satisfies a fluid delivery threshold means that the two liquids together create a risk of fluid overload (e.g., the patient will soon be fluid overloaded). In some implementations, that the sum of the first and second programmed amounts satisfies a fluid delivery threshold means that the two liquids together are insufficient to eliminate a risk of dehydration (e.g., a patient is already dehydrated).

According to some implementations, the medical device operation includes an alert indicating that the sum of the determined first and second programmed amounts satisfies a fluid delivery threshold (e.g., thresholds 214 and 215 of FIG. 2). For example, the alert may be provided at a user interface (e.g., user interface 200 of FIG. 2) of the first or second medical device, an ADM responsible for dispensing the first or second fluid, a total fluid monitoring device, or a system responsible for managing an electronic medical record of a patient. In some implementations, a representation of the alert (e.g., hydration status indicators 211, 241, or 251 of FIG. 2) includes an amount of time (e.g., a countdown timer) after which a patient will be fluid overloaded or dehydrated, which may also be indicated in graphic 213. In some implementations, an alert is provided that there is a risk of dehydration regardless of whether a fluid request was received. For example, in some implementations, a hydration level is determined intermittently, and the alert is generated whenever the hydration level satisfies a fluid delivery threshold.

In some implementations, the process 300 further includes estimating a water loss of a patient with respect to a weight of a patient, a height of the patient, a sex of the patient, a disease state of the patient, a body temperature of the patient, or a body surface area of the patient. In such implementations, the fluid delivery threshold may be based on a threshold amount of water loss before a risk to the patient becomes significant. The water loss of the patient may be relevant to the rate at which fluid can be provided to the patient. For example, if the water loss of the patient is low (e.g., if the patient suffers from anhidrosis), then the amount of liquid that can be safely provided to the patient may decrease. As another example, if the water loss of the patient is high (e.g., if the patient is overweight), then the amount of liquid that can be safely provided to the patient may increase. Indeed, the amount of liquid that must be provided to the patient in order to avoid dehydration may increase, as well.

In some implementations, determining whether the medical device operation should be adjusted includes determining a hydration of the patient. For example, a hydration of the patient may be determined based on measurements from a fluid monitoring system such as a fluid container monitoring system shown and described in U.S. Pat. No. 9,995,619, entitled “Fluid container measurement system employing load cell linkage member,” which is hereby incorporated by reference in its entirety. For example, the catheter-based fluid container monitoring system may measure urine output of the patient and periodically update the system with such measurement information. The system may be in direct communication with the fluid monitoring system (e.g., “paired”), or it may communicate indirectly with the fluid monitoring system via a network or intermediate healthcare information system such as an electronic medical record system. For example, using an identifier for the patient, the system may retrieve information about the patient from the electronic medical record system.

As depicted in FIG. 3, the process 300 continues by adjusting, responsive to the determination (304), the medical device operation based on the fluid delivery request and the first and second programmed amounts. In some implementations, adjusting the medical device operation includes delaying the fluid delivery request. For example, an infusion device 100, on receiving a request to initiate administration of the fluid, may prevent such administration until the clinician acknowledges the alert and confirms the administration and/or until a second clinician acknowledges and confirms the administration. In some implementations, adjusting the medical device operation includes denying the fluid delivery request. For example, if the first medical device is a medicine dispensing machine, the dispensing machine may refuse to dispense the first fluid. In some implementations, the dispensing machine may also inform the user of a risk to the patient (e.g., a risk of fluid overload).

In some implementations, adjusting the medical device operation includes adjusting the first programmed amount. In some implementations, adjusting the medical device operation includes adjusting the second programmed amount. In some implementations, adjusting the medical device operation includes the first or second medical device (e.g., an infusion device 100) adjusting a dilution of the first fluid during administration of the fluid. In some implementations, adjusting the medical device operation includes adjusting a dilution of the second fluid. In some implementations, adjusting the medical device includes recommending to the user an adjustment of the medical device operation via a display associated with the device. For example, in some implementations, the user is recommended to decrease the dilution of the second fluid because otherwise the patient would be at risk for fluid overload. As another example, in some implementations, the user is recommended to mix the first and second fluids (e.g., at a certain dilution rate, at a certain mixing rate, etc.) and provide the two fluids together in a single administration. As yet another example, in some implementations, the user is recommended to decrease one of the first or second amounts of, respectively, the first or second fluids.

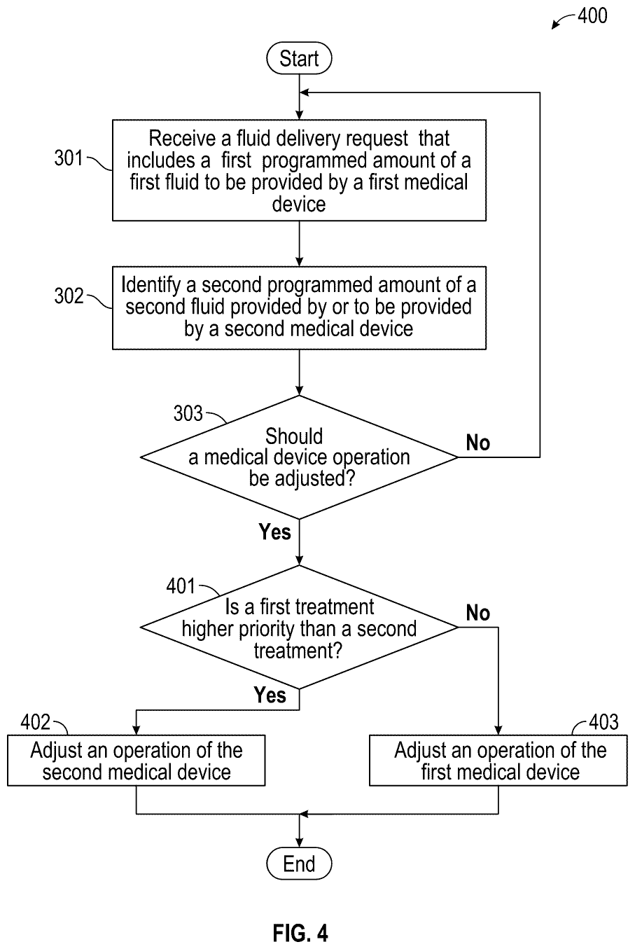

FIG. 4 depicts a second example process 400 for managing patient hydration, according to aspects of the subject technology. According to various implementations, process 400 operates similar to and/or may replace or augment process 300. One or more blocks of process 400 may be implemented, for example, by one or more computing devices including, for example, infusion device 10 of FIG. 1 or processing unit(s) 512 of FIG. 5. In some implementations, one or more of the blocks may be implemented based on one or more machine learning algorithms. In some implementations, one more of the blocks may be implemented apart from other blocks, and by one or more different processors or devices. Further, for explanatory purposes, the blocks of example process 400 are described as occurring in serial, or linearly. However, multiple blocks of example process 400 may occur in parallel. Additionally, the blocks of example process 400 need not be performed in the order shown and one or more of the blocks of example process 400 need not be performed.

As depicted in FIG. 4, in some implementations, the process 400 includes receiving (301) a fluid delivery request that includes a first programmed amount of a first fluid to be provided by a first medical device, identifying (302) a second programmed amount of a second fluid provided by or to be provided by a second medical device, and determining (303) whether a medical device operation should be adjusted. For variations on these steps of the process 400, see the above discussion of process 300.

In the depicted example, the process 400 includes determining (401) whether a first treatment is of higher priority than a second treatment. Depending on the priorities of the respective medication delivery requests, adjustments may be made to accommodate the priorities. For example, a patient may undergo multiple treatments within a short amount of time (e.g., simultaneously). The patient may undergo a first treatment that includes receiving a first amount of a first fluid from a first medical device. The patient may also undergo a second treatment that includes receiving a second amount of a second fluid from a second medical device.

If the first treatment is of higher priority than the second treatment, then the system may determine that adjustments may be made to the second medical device to prioritize the first treatment. For example, if the fluids have not been administered and administration of both fluids would overhydrate the patient then the system may decrease administration of the second fluid. In some implementations, both fluids may be decreased based on amounts weighted based on priority. For example, the first fluid may be decreased less than the second fluid based on the first fluid having a higher priority (and thus greater weighting than the second fluid).

Accordingly, in the depicted example, if it is determined (401-Y) that the first treatment is of higher priority than the second treatment, the process 400 includes adjusting (402) an operation of the second medical treatment. In other words, the second medical treatment may be adjusted when it is the lower-priority treatment. In some implementations, if it is determined (401-N) that the first treatment is not of higher priority than the second treatment, the process 400 includes adjusting (403) an operation of the first medical treatment. In other words, the second medical treatment may not be adjusted when it not the lower-priority treatment.

The foregoing determination of priority (401) allows the process to prioritize higher priority treatments over lower priority treatments. For example, a patient may be receiving a saline solution to maintain hydration, which may constitute a first treatment. After a request for a second treatment is received, it is determined that the second treatment is of higher priority than the first treatment. Whereas the first treatment involved only the administration of saline, the second treatment includes a critical medication. Accordingly, the first treatment may be delayed, cancelled, slowed, or otherwise altered to allow for the second treatment without creating a risk of fluid overload.

Many of the above-described example processes 300, 400, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described herein is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 5 is a conceptual diagram illustrating an example electronic system 500 for managing patient hydration, according to aspects of the subject technology. Electronic system 500 may be implemented by a computing device for execution of software associated with one or more portions or steps of processes 300 and/or 400, or components and methods provided by FIGS. 1-4. In this regard, electronic system 500 may include the infusion device 100, a total fluid monitoring device (e.g., displaying user interface 200 of FIG. 2), and/or a computing device within or connected to the infusion device 100.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, input device interface(s) 514, an output device interface(s) 506, and network interface(s) 516. In some implementations, electronic system 500 may include or be integrated with other computing devices or circuitry for operation of the various components and methods previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) 512 can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is powered off. Some implementations of the subject disclosure use a mass-storage device (such as a magnetic or optical disk and its corresponding disk drive) as the permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as random-access memory (RAM). System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process, in order to execute the processes of some implementations.

Bus 508 also connects to input device interface(s) 514 and output device interface(s) 506. Input device interface(s) 514 enable the user to communicate information and select commands to the electronic system. Input devices used with input device interface(s) 514 include, for example, alphanumeric keyboards and pointing devices (also called “cursor control devices”). Output device interface(s) 506 enable, for example, the display of images generated by the electronic system 500. Output devices used with output device interface(s) 506 include, for example, printers and display devices, such as cathode ray tubes (CRT) or liquid crystal displays (LCD). Some implementations include devices such as touchscreens that functions as both input and output devices.

Also, as shown in FIG. 5, bus 508 also couples electronic system 500 to a network (not shown) through network interface(s) 516. Network interface(s) 516 may include, for example, a wireless access point (e.g., Bluetooth or Wi-Fi) or radio circuitry for connecting to a wireless access point. Network interface(s) 516 may also include hardware (e.g., ethernet hardware) for connecting the computer to a part of a network of computers such as a local area network (LAN), a wide area network (WAN), wireless LAN, an intranet, or a network of networks, such as the Internet. Any or all components of electronic system 500 can be used in conjunction with the subject disclosure when specifically configured with one of more of the features described.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices specifically configured with one or more of the fluid tracking features described. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in forms such as acoustic, speech, gesture, or tactile input. In addition, a computer can interact with a user by sending documents to and receiving documents from a device that is used by the user, e.g., by sending web pages to a web browser on a user's client device in response to requests received from the web browser.

Implementations of the subject matter described in this specification can be implemented in a specifically configured computing system that includes a back end component, e.g., as a data server, or that includes a specifically configured middleware component, e.g., an application server, or that includes a specifically configured front end component, e.g., a client computer having a graphical user interface or a Web browser through which a user can interact with an implementation of the subject matter described in this specification, or any combination of one or more such back end, middleware, or front end components. The components of the system can be interconnected by one or more forms or mediums of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for managing patient hydration, comprising: receiving a fluid delivery request that includes a first programmed amount of a first fluid to be provided by a first medical device; identifying a second programmed amount of a second fluid provided by or to be provided by a second medical device; determining, based on the first and second programmed amounts, that a medical device operation should be adjusted; and adjusting, responsive to the determination, the medical device operation based on the fluid delivery request and the first and second programmed amounts.

Clause 2. The method of Clause 1, wherein adjusting the medical device operation comprises delaying or denying the fluid delivery request.

Clause 3. The method of Clause 1, wherein adjusting the medical device operation comprises adjusting the first programmed amount or the second programmed amount.

Clause 4. The method of Clause 1, wherein adjusting the medical device operation comprises adjusting a dilution of the first fluid or a dilution of the second fluid.

Clause 5. The method of Clause 1, wherein adjusting the medical device operation comprises denying a dispensation of the first fluid by a dispensing machine.

Clause 6. The method of any one of Clauses 1 through 5, wherein the first medical device comprises a first infusion pump configured to provide the first programmed amount of the first fluid over a first programmed period of time.

Clause 7. The method of any one of Clauses 1 through 6, wherein the second medical device comprises a second infusion pump configured to provide the second amount of the second fluid over a second programmed period of time.

Clause 8. The method of any one of Clauses 1 through Clause 7, wherein adjusting the medical device operation comprises adjusting a duration of time programmed into a respective infusion pump for administering a respective fluid to a patient.

Clause 9. The method of any one of Clauses 1 through 8, wherein determining that the medical device operation should be adjusted comprises determining that a sum of the first and second programmed amounts satisfies a fluid delivery threshold.

Clause 10. The method of Clause 9, wherein adjusting the medical device operation comprises providing an alert indicating that the sum of the first and second programmed amounts satisfies the fluid delivery threshold.

Clause 11. The method of Clause 9 or Clause 10, further comprising: estimating a water loss of a patient with respect to a weight of the patient, a height of the patient, a sex of the patient, a disease state of the patient, a body temperature of the patient, or a body surface area of the patient; and wherein the fluid delivery threshold is based on the estimated water loss of the patient.

Clause 12. The method of Clause 11, further comprising: receiving a hydration of the patient; and wherein the fluid delivery threshold is based on the hydration of the patient.

Clause 13. The method of Clause 12, wherein receiving the hydration of the patient comprises receiving the hydration information for the patient from a catheter system.

Clause 14. The method of any one of Clauses 1 through 13, further comprising: determining that a first treatment is of higher priority than a second treatment, wherein the first treatment comprises providing, by the first medical device, the first programmed amount of the first fluid and the second treatment comprises providing, by the second medical device, the second programmed amount of the second programmed amount of the second fluid; and wherein adjusting the medical device operation comprises adjusting an operation of the second medical device.

Clause 15. The method of any one of Clauses 1 through 13, further comprising: determining that a second treatment is of higher priority than a first treatment, wherein the first treatment comprises providing, by the first medical device, the first programmed amount of the first fluid and the second treatment comprises providing, by the second medical device, the second programmed amount of the second fluid; and wherein adjusting the medical device operation comprises adjusting an operation of the first medical device.

Clause 16. The method of any one of Clauses 1 through 15, wherein the fluid delivery request comprises a medication order for delivery of a medication to a patient by the first medical device.

Clause 17. The method of any one of Clauses 1 through 16, wherein the first medical device comprises a first infusion pump configured to administer the first fluid to a patient, the second medical device comprises a second infusion pump configured to administer the second fluid to the patient, the second fluid comprises a saline solution, and adjusting the medical device operation comprises automatically decreasing the second programmed amount.

Clause 18. The method of any one of Clauses 1 through 15, wherein the first medical device comprises an automated dispensing machine, and the fluid delivery request includes a request to dispense the first fluid from the dispensing machine.

Clause 19. An electronic device, comprising: one or more processors; and memory storing one or more programs, the one or more programs including instructions, which, when executed by the one or more processors, cause the electronic device to perform the method of any of Clauses 1-18.

Clause 20. A non-transitory, computer-readable storage medium storing one or more programs that include instructions which, when executed by the one or more processors of an electronic device, cause the electronic device to perform the method of any of Clauses 1-18.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to,” “operable to,” and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic,” as used herein, may include performance by a computer or machine without user intervention, for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network-based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware clement without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fu5y logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A method for managing patient hydration, comprising:
receiving a fluid delivery request that includes a first programmed amount of a first fluid to be provided by a first medical device;
identifying a second programmed amount of a second fluid provided by or to be provided by a second medical device;
determining, based on the first and second programmed amounts, that a medical device operation should be adjusted; and
adjusting, responsive to the determination, the medical device operation based on the fluid delivery request and the first and second programmed amounts.

2. The method of claim 1, wherein adjusting the medical device operation comprises delaying or denying the fluid delivery request.

3. The method of claim 1, wherein adjusting the medical device operation comprises adjusting the first programmed amount or the second programmed amount.

4. The method of claim 1, wherein adjusting the medical device operation comprises adjusting a dilution of the first fluid or a dilution of the second fluid.

5. The method of claim 1, wherein adjusting the medical device operation comprises denying a dispensation of the first fluid by a dispensing machine.

6. The method of claim 1, wherein the first medical device comprises a first infusion pump configured to provide the first programmed amount of the first fluid over a first programmed period of time.

7. The method of claim 1, wherein the second medical device comprises a second infusion pump configured to provide the second programmed amount of the second fluid over a second programmed period of time.

8. The method of claim 1, wherein adjusting the medical device operation comprises adjusting a duration of time programmed into a respective infusion pump for administering a respective fluid to a patient.

9. The method of claim 1, wherein determining that the medical device operation should be adjusted comprises determining that a sum of the first and second programmed amounts satisfies a fluid delivery threshold.

10. The method of claim 9, wherein adjusting the medical device operation comprises providing an alert indicating that the sum of the first and second programmed amounts satisfies the fluid delivery threshold.

11. The method of claim 9, further comprising:
estimating a water loss of a patient with respect to a weight of the patient, a height of the patient, a sex of the patient, a disease state of the patient, a body temperature of the patient, or a body surface area of the patient; and
wherein the fluid delivery threshold is based on the estimated water loss of the patient.

12. The method of claim 11, further comprising:
receiving a hydration of the patient; and
wherein the fluid delivery threshold is based on the hydration of the patient.

13. The method of claim 12, wherein receiving the hydration of the patient comprises receiving the hydration of the patient from a catheter system.

14. The method of claim 1, further comprising:
determining that a first treatment is of higher priority than a second treatment, wherein the first treatment comprises providing, by the first medical device, the first programmed amount of the first fluid and the second treatment comprises providing, by the second medical device, the second programmed amount of the second fluid; and
wherein adjusting the medical device operation comprises adjusting an operation of the second medical device.

15. The method of claim 1, further comprising:
determining that a second treatment is of higher priority than a first treatment, wherein the first treatment comprises providing, by the first medical device, the first programmed amount of the first fluid and the second treatment comprises providing, by the second medical device, the second programmed amount of the second fluid; and
wherein adjusting the medical device operation comprises adjusting an operation of the first medical device.

16. The method of claim 1, wherein the fluid delivery request comprises a medication order for delivery of a medication to a patient by the first medical device.

17. The method of claim 1, wherein the first medical device comprises a first infusion pump configured to administer the first fluid to a patient, the second medical device comprises a second infusion pump configured to administer the second fluid to the patient, the second fluid comprises a saline solution, and adjusting the medical device operation comprises automatically decreasing the second programmed amount.

18. The method of claim 1, wherein the first medical device comprises an automated dispensing machine, and the fluid delivery request includes a request to dispense the first fluid from the automated dispensing machine.

19. A system, comprising:
a first medical device;
a second medical device; and
an electronic device that includes one or more processors and memory storing one or more programs, the one or more programs including instructions, which, when executed by the one or more processors, cause the electronic device to perform operations comprising:
receiving a fluid delivery request that includes a first programmed amount of a first fluid to be provided by the first medical device;
identifying a second programmed amount of a second fluid provided by or to be provided by the second medical device;
determining, based on the first and second programmed amounts, that a medical device operation should be adjusted; and
adjusting, responsive to the determination, the medical device operation based on the fluid delivery request and the first and second programmed amounts.

20. A non-transitory, computer-readable storage medium storing one or more programs that include instructions which, when executed by one or more processors of an electronic device, cause the electronic device to perform operations comprising:
receiving a fluid delivery request that includes a first programmed amount of a first fluid to be provided by a first medical device;
identifying a second programmed amount of a second fluid provided by or to be provided by a second medical device;
determining, based on the first and second programmed amounts, that a medical device operation should be adjusted; and
adjusting, responsive to the determination, the medical device operation based on the fluid delivery request and the first and second programmed amounts.
